(19) United States
(12) Patent Application Publication
(10) Pub. No.: US 2023/0310163 A1
(43) Pub. Date: Oct. 5, 2023

(54) FEMORAL ORTHOPEDIC IMPLANT OF A KNEE PROSTHESIS

(71) Applicants: Rajesh MANIAR, Mumbai, Maharashtra (IN); Adit MANIAR, Mumbai, Maharashtra (IN)

(72) Inventors: Rajesh MANIAR, Mumbai, Maharashtra (IN); Adit MANIAR, Mumbai, Maharashtra (IN)

(21) Appl. No.: 18/023,284

(22) PCT Filed: Aug. 27, 2021
(86) PCT No.: PCT/IB2021/057859

§ 371 Date: Feb. 24, 2023

(30) Foreign Application Priority Data
Aug. 29, 2020 (IN) 202021037276

(51) Int. Cl. A61F 2/38

(52) U.S. Cl. CPC A61F 2/3859; A61F 2/3877; A61F 2/389

(57) ABSTRACT

The present disclosure discloses a femoral orthopedic implant (8) of a knee prosthesis (100). The femoral orthopedic implant (8) includes a condylar member (12) and a posterior flange (2) defined by a predetermined length extending away from one end of the condylar member (12). Further, the femoral orthopedic implant (8) includes an anterior flange (1) extending from another end, opposite and away from the one end of the condylar member (12), where the anterior flange (1) is defined with a length ranging from about 0.75 to 1.4 times the predetermined length of the posterior flange (2).

### TECHNICAL FIELD

Present disclosure in general, relates to a field of medical devices. Particularly, but not exclusively, the present disclosure relates to a knee prosthesis. Further, embodiments of the present disclosure disclose a femoral orthopedic implant of the knee prosthesis.

### BACKGROUND OF THE DISCLOSURE

Generally, human knee joints endure exceptional loads and a wide variety of loading scenarios throughout the life of an individual. While the human knee joint supports most of these typical loads for entire life of the individual, in certain circumstances the human knee joint may degrade and performance of the knee joint may decline. For example, injury to the knee causes damage to the knee joint which may not fully recover naturally or even with medical intervention. In such circumstances, it is beneficial to replace the natural knee joint with an artificial knee joint. Further, any degenerative disease suffered by the individual can act on the natural knee joint to degrade the performance in an irreversible fashion, such that replacement of the natural knee joint with an artificial knee joint is inevitable.

Typically, artificial knee joints are well known and have come into widespread use. Conventionally, the artificial knee joints include a femoral component, a tibial component, a patellar component and a tibial liner connected to the tibial component and positioned between the femoral component and the tibial component. A distal end of a femur is surgically prepared to have a profile matching the profile of the femoral component. The femoral component is then attached to the distal end of the femur with or without cement. Similarly, a proximal end of a tibia is prepared, typically by cutting a flat proximal surface on the proximal end of the tibia, and the tibial component is attached to the flat surface formed on the proximal end of the tibia. The tibial liner provisioned over the surface of the tibial component is configured to glide over distal surfaces of the femoral component and is designed to facilitate articulation relative to each other in the same way that the distal end of a natural femur articulates relative to the proximal end of a natural tibia. However, the conventional artificial knee joints are complex to implant, as the femoral component requires the distal end of the femur to be extensively shaped to properly mate and securely lock onto the distal end of the femur. Further, shaping of the femur requires skill and is a complex and a tedious process as cuts have to be made to correspond to the profile of the femoral component. Moreover, different individuals have different sizes of femur and various femoral components having different sizes must be considered before selecting the particular femoral component. Furthermore, for shaping the femur, specialized cutting jigs are employed which are expensive and difficult to maintain.

FIGS. 1a-1b illustrates side views of conventional [existing art] femoral component (8a) and the conventional femoral component accommodated at a distal end of the femur (21), respectively. FIG. 3 illustrates a conventional knee prosthesis which replaces biological part of the knee that comprises a patella (22), a tibia (23), a femoral condyle (femur (21)) and an anterior femoral fat pad (9). The conventional femoral component (8a) includes an anterior flange (1a) and a posterior condyle (2a), where the anterior flange (1a) has a long standard flange length and a broad edge (3). With advancements in technology, the broad edge (3) of the conventional anterior flange (1a) is narrowed and smoothened to form an angular structure which results in a long tapered (4) anterior flange (1a). The taper (4) provides a clearance between the conventional femoral component (8a) and tissue to reduce contact with tissues. However, friction is not completely eliminated since the anterior flange (1a) length is such that, the anterior flange (1a) extends beyond the natural anterior articular cartilage of the femur (21). Also, in providing for such anterior flange (1a) to sit there, one needs to compromise and excise the anterior femoral fat pad (9), which in natural knee provides cushioning and eliminates friction. Due to the friction, the femur (21) or structure coupled to the femur (21) in the conventional artificial knee joints abut the patella (22) or structures coupled to the patella (22) and prevent further flexion which prevents large amounts of flexion to replicate the operations of the natural human knee joint. In view of the same, the conventional knee prosthesis generates friction at designated frictional contact areas (10) between the components and the soft tissues during full extension, and at mid flexion and 90° flexion of the knee, thereby resulting in stress generation at the knee joint which may cause discomfort and pain to the user and may limit deep flexion.

The present disclosure is directed to overcome one or more limitations stated above or any other limitations associated with the existing arts.

### SUMMARY OF THE DISCLOSURE

One or more shortcomings of the prior art are overcome by a femoral orthopedic implant and a knee prosthesis as claimed and additional advantages are provided through the femoral orthopedic implant and the knee prosthesis as claimed in the present disclosure. Additional features and advantages are realized through the techniques of the present disclosure. Other embodiments and aspects of the disclosure are described in detail herein and are considered a part of the claimed disclosure.

In one non-limiting embodiment of the present disclosure, a femoral orthopedic implant of a knee prosthesis is disclosed. The femoral orthopedic implant includes a condylar member and a posterior flange which is defined by a predetermined length extending away from one end of the condylar member. Further, the femoral orthopedic implant includes an anterior flange extending from another end, opposite and away from the one end of the condylar member, where the anterior flange is defined with a length ranging from about 0.75 to 1.4 times the predetermined length of the posterior flange.

In an embodiment, length of the anterior flange is equal to the predetermined length of the posterior flange.

In an embodiment, the femoral orthopedic implant includes a groove extending along a length of the condylar member and the anterior flange.

In an embodiment, the posterior flange is defined with a pair of posterior flange attachment members extending parallel to each other from the condylar member.

In an embodiment, wherein the condylar member, the posterior flange and the anterior flange are joined to form a unitary structure.

In an embodiment, the anterior flange and the posterior flange extend integrally from the condylar member defining a C-shaped or an U-shaped femoral orthopedic implant.

In an embodiment, the condylar member, the posterior flange and the anterior flange comprises a first surface defined with a plurality of chamfers corresponding to a profile of a femur.

In an embodiment, the femoral orthopedic implant includes at least one projection configured to secure and lock the end of the femur and the femoral orthopedic implant.

In another non-limiting embodiment of the present disclosure a knee prosthesis is disclosed. The knee prosthesis includes a tibia orthopedic implant which is connectable to a top end of a tibia and a patella orthopedic implant connectable to a patella. Further, the knee prosthesis includes a femoral orthopedic implant which is connectable to an end of a femur. The femoral orthopedic implant includes a condylar member and a posterior flange which is defined by a predetermined length extending away from one end of the condylar member. Further, the femoral orthopedic implant includes an anterior flange extending from another end, opposite and away from the one end of the condylar member, where the anterior flange is defined with a length ranging from about 0.75 to 1.4 times the predetermined length of the posterior flange.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE ACCOMPANYING DRAWINGS

The novel features and characteristics of the disclosure are set forth in the appended claims. The disclosure itself, however, as well as a preferred mode of use, further objectives, and advantages thereof, will best be understood by reference to the following detailed description of an illustrative embodiments when read in conjunction with the accompanying figures. One or more embodiments are now described, by way of example only, with reference to the accompanying figures wherein like reference numerals represent like elements and in which:

FIGS. 1a and 1b illustrate illustrates a side views of conventional femoral component and the conventional femoral component accommodated at a distal end of the femur, respectively (prior art).

FIGS. 2(a), 2(b) and 2(c) illustrate sectional side views illustrating anatomy of a knee with areas of friction during full extension, and at mid flexion and 90° flexion, of the knee joint.

FIGS. 3(a), 3(b) and 3(c) illustrates sectional side views of the positions of a conventional knee prosthesis during full extension, mid flexion and 90° flexion of a knee joint.

FIGS. 4(a), 4(b) and 4(c) illustrate a sectional side views of a knee prosthesis indicating reduction in areas of friction during full extension, at mid flexion and 90° flexion of the knee joint, in accordance with an embodiment of the present disclosure.

FIG. 5(a) illustrates a perspective view of the femoral orthopedic implant, in accordance with an embodiment of the present disclosure.

FIGS. 5(b), 5(c) and 5(d) illustrate a side view of a femoral orthopedic implant, in accordance with an embodiment of the present disclosure.

FIG. 6 illustrates a sectional side view of a femoral orthopedic implant connected to an end of a femur, in accordance with an embodiment of the present disclosure.

FIG. 7 illustrates an axial cross section of femoral articulation in (a) conventional femoral orthopedic implant; (b) conventional femoral orthopedic implant with smoother borders; (c) conventional femoral orthopedic implant with reduced height; (d) conventional femoral orthopedic implant with narrow profile; and (e) exemplary embodiment of the present disclosure, where the femoral orthopedic implant enables articulation with fat pad.

FIG. 8 illustrates a anterior surface of the femoral orthopedic implant for (a) conventional femoral orthopedic implant for both sides; (b) conventional femoral orthopedic implant with side specific shape; (c) conventional femoral orthopedic implant with narrow condylar member; (d) exemplary embodiment of the present disclosure.

FIG. 9 illustrates a groove in coronal plane for the femoral orthopedic implant, in accordance with an embodiment of the present disclosure.

The figures depict embodiments of the disclosure for purposes of illustration only. One skilled in the art will readily recognize from the following description that alternative embodiments of the system and method illustrated herein may be employed without departing from the principles of the disclosure described herein.

### DETAILED DESCRIPTION

The foregoing has broadly outlined the features and technical advantages of the present disclosure in order that the detailed description of the disclosure that follows may be better understood. Additional features and advantages of the disclosure will be described hereinafter which form the subject of the claims of the disclosure. It should be appreciated by those skilled in the art that, the conception and specific embodiments disclosed may be readily utilized as a basis for modifying other femoral orthopedic implant and a knee prosthesis for carrying out the same purposes of the present disclosure. It should also be realized by those skilled in the art that, such equivalent constructions do not depart from the scope of the disclosure as set forth in the appended claims. The novel features which are believed to be characteristics of the disclosure, together with further objects and advantages will be better understood from the following description when considered in connection with the accompanying figures. It is to be expressly understood, however, that each of the figures is provided for the purpose of illustration and description only and is not intended as a definition of the limits of the present disclosure.

The terms “comprises”, “comprising”, or any other variations thereof, are intended to cover a non-exclusive inclusions, such that a system or a device that comprises a list of components or steps does not include only those components or steps but may include other components or steps does not expressly listed or inherent to such setup or device or method. In other words, one or more elements in the system or apparatus proceeded by “comprises . . . a” does not, without more constraints, preclude the existence of other elements or additional elements in the system or apparatus.

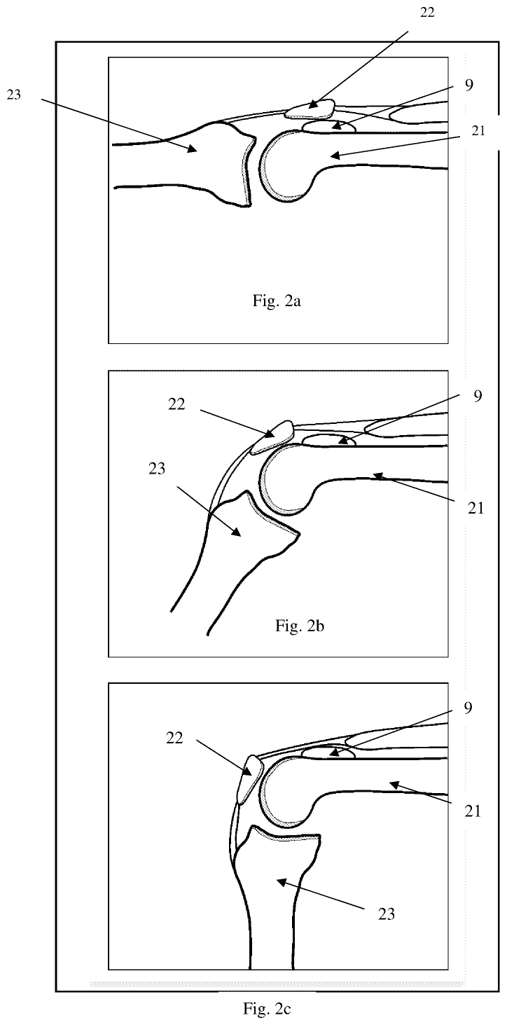

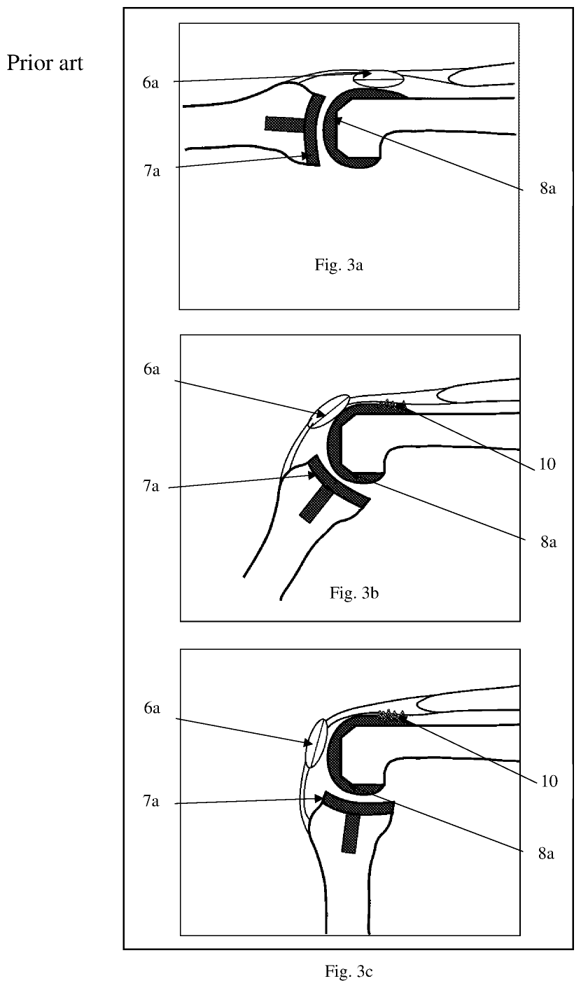

FIG. 2 illustrates a natural knee joint with views of a normal anatomy of a knee, depicting areas of friction during full extension, at mid flexion and 90° flexion of the knee. FIG. 3 illustrates a conventional knee prosthesis which replaces biological part of the knee that comprises a patella (22), a tibia (23), a femoral condyle (femur (21)) and an anterior femoral fat pad (9). The conventional knee prosthesis includes conventional patella orthopedic implant (6a) that replaces the back of the patella (22) or the knee cap, the conventional tibia orthopedic implant (7a) is provided on the top surface of the tibia (23) and the conventional femoral orthopedic implant (8a) is received at the distal end of the femur (21). Further, the conventional knee prosthesis generates friction at designated frictional contact areas (10) between the implants and the soft tissues thereby resulting in stress generation at the knee joint which may cause discomfort and pain to the user.

Accordingly, the present disclosure discloses a knee prosthesis. The knee prosthesis includes a tibia orthopedic implant which is connectable to a top end of a tibia, an insert connectable to the tibia orthopedic implant and a patella orthopedic implant connectable to a patella. Further, the knee prosthesis includes a femoral orthopedic implant which is connectable to an end of a femur. The femoral orthopedic implant includes a condylar member and a posterior flange which is defined by a predetermined length extending away from one end of the condylar member. Further, the femoral orthopedic implant includes an anterior flange extending from another end, opposite and away from the one end of the condylar member, where the anterior flange is defined with a length ranging from about 0.75 to 1.4 times the predetermined length of the posterior flange. The configuration of the femoral orthopedic implant enables the knee prosthesis to match normal knee anatomy and eliminate the irritation to the muscles and soft tissues around the knee prosthesis.

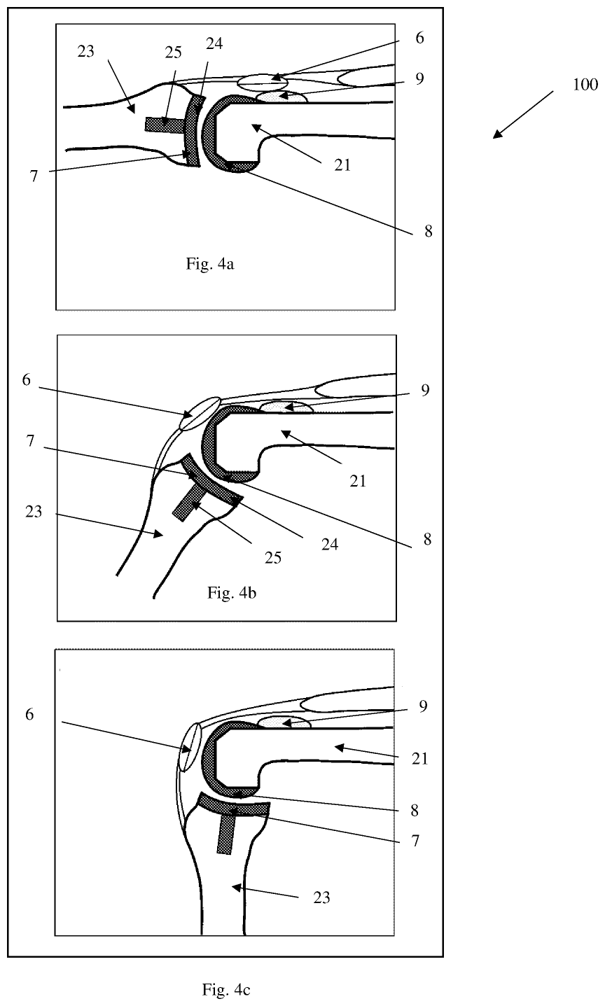

Reference will now be made to the exemplary embodiments of the disclosure, as illustrated in the accompanying drawings. Wherever possible, same numerals have been used to refer to the same or like parts. The following paragraphs describe the present disclosure with reference to FIGS. 4-8.

FIGS. 4a, 4b and 4c are exemplary embodiments of the present disclosure, which illustrates a schematic view of a knee prosthesis (100) accommodated in a knee of a subject. The knee prosthesis (100) may include a tibia orthopedic implant (7) which may be connectable to a top end of a tibia (23). The tibia orthopedic implant (7) may include a flat plate (24), which may be configured to connect with the top surface of the tibia (23). Further, the tibia orthopedic implant (7) may include a protrusion (25) extending from a portion of the flat plate (24) into the tibia (23). The protrusion (25) may be configured to be insertable into the tibia (23) through the top surface. As apparent from FIGS. 4a-4c, the knee prosthesis (100) may include a patella orthopedic implant (6) connectable to a patella (22) and a femoral orthopedic implant (8), which may be connectable to an end of a femoral condyle of the femur (21). Furthermore, an insert or a tibial liner [not shown in figs] may be connected to the tibia orthopedic implant (7) and provisioned between the tibia orthopedic implant (7) and femoral orthopedic implant (8) which may be configured to aid gliding of the tibia orthopedic implant (7) over the femoral orthopedic implant (8). In an embodiment, the insert may be made of flexible material which may be resilient than that material of the tibial orthopedic implant. The insert may be configured to duplicate the function of a natural meniscus within a natural knee joint and may help in minimizing friction in the articulation of the femoral orthopedic implant (8) relative to the tibial orthopedic implant. In an embodiment, the femoral orthopedic implant (8) may be defined with a shape resembling the shape of the femoral condyle of the femur (21) such that, the femoral orthopedic implant (8) may receive a distal end of the femur (21). The tibia orthopedic implant (7) may be structured to glide along a surface of the femoral orthopedic implant (8).

In an embodiment, the terms anterior and posterior may be inferred as nearer the front and near the back of the body of the object being referred to, respectively. Thus, for the knee joint described herein, anterior refers to that portion of the knee that is nearer to the front of the body when the leg is in an extended position. Proximal and distal respectively mean nearer to and further away from the root of the structure in question. For example, the distal end of the femur is the end of the femur that forms part of the knee joint and the proximal end of the femur is the end of the femur that forms part of the hip joint.

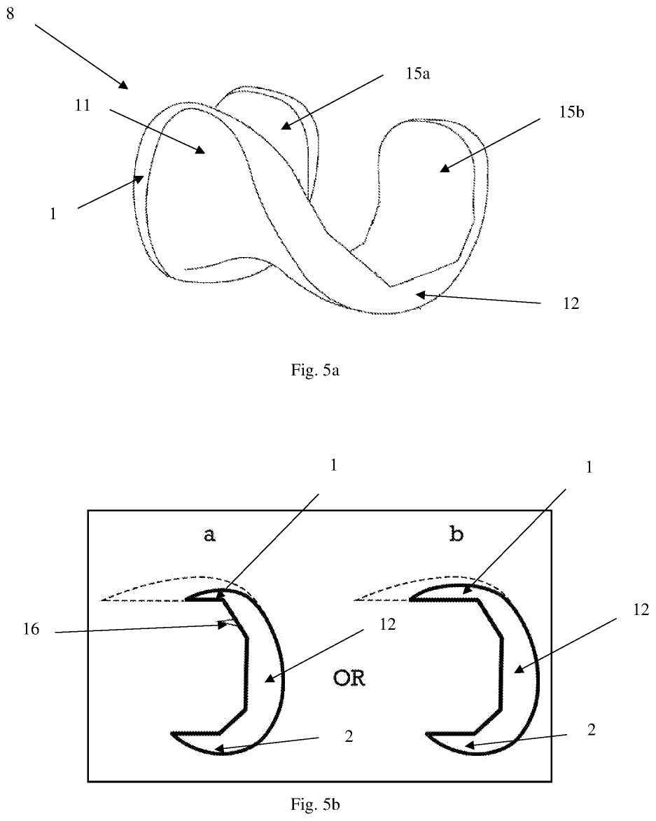

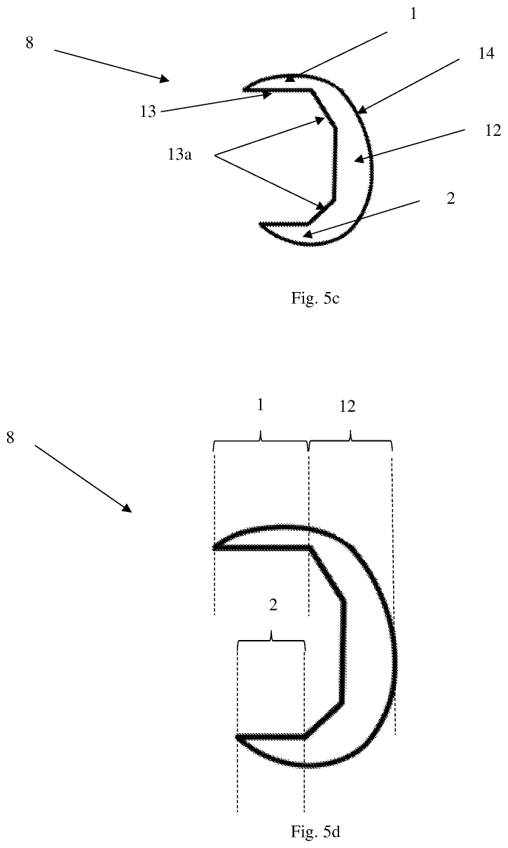

FIGS. 5a, 5b, 5c and 5d are exemplary embodiments of the present disclosure, which illustrate perspective view and side views of the femoral orthopedic implant (8). The femoral orthopedic implant (8) may include a condylar member (12) and a posterior flange (2) extending away from one end of the condylar member (12). The posterior flange (2) may be defined by a predetermined length. In an embodiment, the predetermined length of the posterior flange (2) may be based on the dimensions of the distal end of the femur (21) onto which the femoral orthopedic implant (8) may be positioned. Further, as seen in FIG. 5a the posterior flange (2) may be defined with a pair of posterior flange attachment members (15a, 15b) extending parallel to each other from the condylar member (12). In an illustrated embodiment, the posterior flange (2) includes two posterior flange attachment members (15a, 15b), however the same should not construed as a limitation as the posterior flange (2) may be defined with more or less than two posterior flange attachment members (15a, 15b). As seen in FIGS. 5b-5d, the femoral orthopedic implant (8) may further include an anterior flange (1) which may extend from another end, opposite to the one end of the condylar member (12). The femoral orthopedic implant (8) may define a cavity between the anterior flange (1) and the posterior flange (2), such that the distal end of the femur (21) may be received within the cavity. In an embodiment, the anterior flange (1) may be defined with a length ranging from about 0.75 to 1.4 times the predetermined length of the posterior flange (2). In an embodiment, the length of the anterior flange (1) may be equal to the predetermined length of the posterior flange (2) or less than predetermined length of the posterior flange (2).

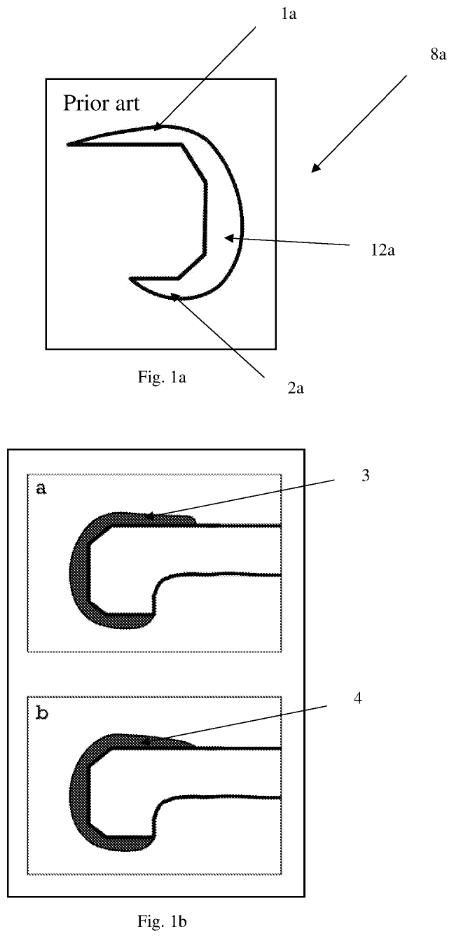

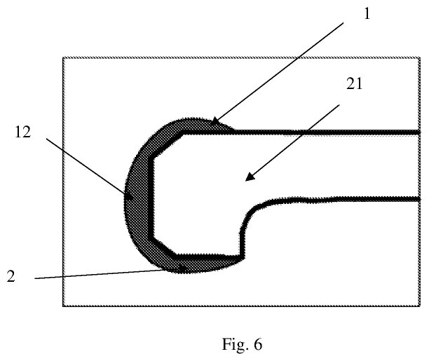

In an embodiment, the length of the anterior flange (1) may be defined to be in the range of 0.75 to 1.4 times the predetermined length of the posterior flange (2), that may result in elimination of friction between the femoral orthopedic implant (8) and the soft tissues as the anterior flange length does not extend much beyond the natural anterior articular cartilage of the femur (21) [as seen in FIG. 6]. This configuration prevents any irritation or discomfort to the user and allows complete and deep flexion of the knee joint, unlike conventional knee prosthesis where the conventional anterior flange (1a) frictionally contacts the soft tissues which causes irritation and discomfort [as seen in FIGS. 1 and 3]. The length of the anterior flange (1) in the range of 0.75 to 1.4 times the predetermined length of the posterior flange (2), when compared to the conventional anterior flange (1a) eliminates the stress formed on the soft tissues surrounding the knee prostheses in the static position and particularly when any flexion-extension movement occurs. Further, the length of the anterior flange (1) also preserves anterior femoral fat pad (9) [hereafter referred to as fat pad (9)] and enables the fat pad (9) to function normally thereby reducing friction and potentially eliminating the irritation to the muscles and soft tissues around.

In an embodiment, the anterior flange (1) may be defined with a smooth surface defined with a slender profile to prevent friction with the soft tissues surrounding the knee prosthesis (100).

Referring back to FIGS. 4a, 4b and 4c which illustrates sectional side views of the knee prosthesis (100) with the femoral orthopedic implant (8) having the anterior flange (1) with the length ranging from about 0.75 to 1.4 times the predetermined length of the posterior flange (2). As seen in FIGS. 4a, 4b and 4c the areas of friction during full extension, and at mid flexion and 90° flexion of the knee may be minimized or prevented. The introduction of substantially smaller anterior flange (1) length as shown in figure causes reduction in frictional contact area (10) of the tissues with the knee prosthesis (100). As seen in FIG. 4c, the fat pad (9) continues to remain in position with more distal extent towards the tibia (23) due to the length of the anterior flange (1) being in the range of 0.75 to 1.4 times the predetermined length of the posterior flange (2) of the femoral orthopedic implant (8).

In an embodiment, the anterior flange (1) and the posterior flange (2) extends integrally from the condylar member (12) such that the femoral orthopedic implant (8) forms a unitary structure.

Further, femoral orthopedic implant (8) may define including but not limited to a C-shaped structure, U-shaped structure and any other shaped structure capable of receiving the distal end of the femur (21).

In an embodiment, the femoral orthopedic implant (8) formed by the condylar member (12), the posterior flange (2) and the anterior flange (1) may be defined with a first surface (13) adapted to abut with the distal end of the femur (21). The first surface (13) may be defined with a plurality of chamfers corresponding to a profile of a femur (21), thereby eliminating extensive shaping of the femur (21). In an embodiment, the first surface (13) of the anterior flange (1) and the posterior flange (2) may be flat and may be shaped to receive and bear against an anterior section of the distal end of the femur (21). Furthermore, the femoral orthopedic implant (8) may be defined with a second surface (14) opposite to the first surface (13). The second surface (14) may be oriented towards the tibia orthopedic implant (7) and may be defined with a profile or a surface pattern suitable for gliding the insert positioned or connected on the tibia orthopedic implant (7) over the femoral orthopedic implant (8).

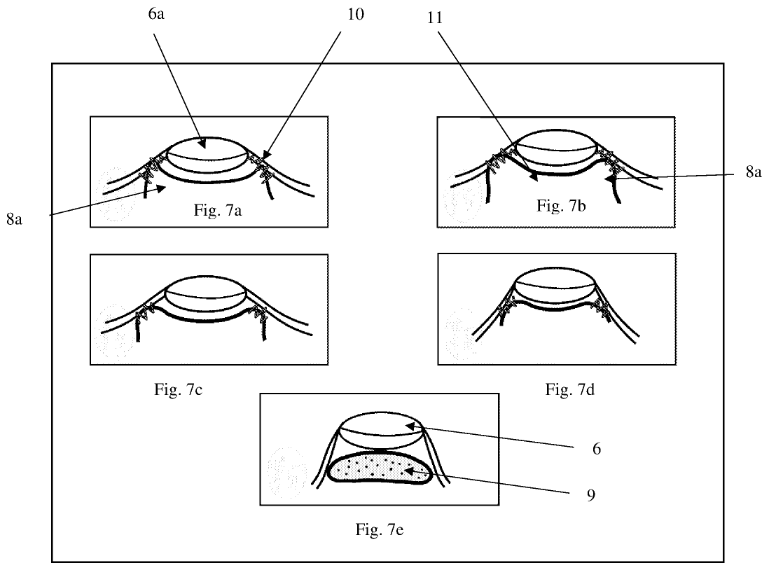

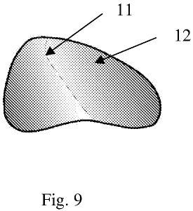

Further, as seen in FIGS. 5d and 7, the femoral orthopedic implant (8) may be defined with a groove (11) that may extending along a length of the condylar member (12) and the anterior flange (1). The groove (11) may be configured to conform with the shape of the distal end of the femur (21). In an embodiment, the groove (11) may be configured to aid in gliding the patella orthopedic implant (6) over the femoral orthopedic implant (8). In an embodiment, as seen in FIG. 9, condylar member (12) may be defined with reduced length, where the width can be profiled as per requirement such that the groove (11) orientation in a coronal plane may be correspondingly modified. In an embodiment, the femoral orthopedic implant (8) may be provided with at least one projection (16) configured to secure and lock the end of the femur (21) and the femoral orthopedic implant (8). The at least one projection (16) may be provided on the first surface (13) of the femoral orthopedic implant (8) at predetermined locations based on locking of the femoral orthopedic implant (8) with the femur (21).

Referring to FIG. 7 that illustrates an axial cross section of knee prosthesis (100) articulation. FIG. 7e refers to an exemplary embodiment of the present disclosure where during articulation of the knee prosthesis (100), the patella orthopedic implant (6), glides over the groove (11) defined on the condylar member (12) and the anterior flange (1) of the femoral orthopedic implant (8). Due to the length of the anterior flange (1) ranging from about 0.75 to 1.4 times the predetermined length of the posterior flange (2) and also due to the narrowed and thin profile of the femoral orthopedic implant (8), the femoral orthopedic implant (8) may not extend to a level where it may interfere with the fat pad (9) thereby eliminating frictional contact area (10). Whereas in comparison, FIG. 7a refers to a conventional femoral orthopedic implant (8a), FIG. 7b refers to the conventional femoral orthopedic implant (8a) with smooth borders, FIG. 7c refers to the conventional femoral orthopedic implant (8a) designed with reduced condylar member (12) height and FIG. 7d refers to a conventional narrowed femoral orthopedic implant (8a) which resulting in a frictional contact area (10) between the knee prosthesis and the tissue surrounding the knee prosthesis.

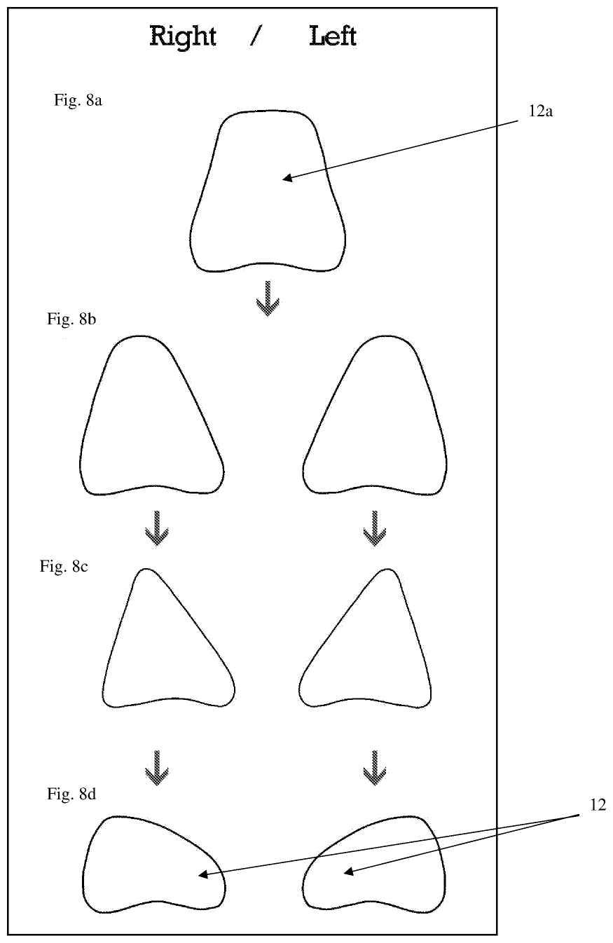

Turning to FIG. 8 which illustrates the shape of an anterior surface of the femoral orthopedic implant (8) in the coronal view. FIG. 8d illustrates the condylar member (12) in accordance with an exemplary embodiment of the present disclosure defined with reduced length, where the width may be reduced and made into a narrowed profile as per requirement. The reduced dimensions aid in preventing contact between the femoral orthopedic implant (8) and the tissue thereby mitigating any discomfort or irritation. Consequently, the fat pad (9) may extend distally towards the patella (22) due to length of the anterior flange (1) being in the range of 0.75 to 1.4 times the predetermined length of the posterior flange (2). This configuration restricts frictional contact area (10) of the soft tissues with the knee prosthesis (100), thereby eliminating discomfort and pain to the user. Whereas FIG. 8a illustrates a conventional condylar member (12a) profile for both sides, FIG. 8b illustrates the conventional condylar member (12a) profile with side specific shape, FIG. 8c illustrates the side specific condylar member (12a) with narrowed profile which resulting in a frictional contact area (10) between the knee prosthesis and the tissue surrounding the knee prosthesis.

In an embodiment, the tibia orthopedic implant (7), the insert, the patella orthopedic implant (6) and the femoral orthopedic implant (8) may be made of any suitable biomaterial having the mechanical properties necessary to function as a human knee. For example, the tibia orthopedic implant (7), the insert, the patella orthopedic implant (6) and the femoral orthopedic implant (8) may be made of materials such as but not limited to metals like titanium, titanium alloy, cobalt chrome alloy, stainless steel and the like, ceramics and high strength polymers.

In an embodiment, the configuration of the femoral orthopedic implant (8) enables the knee prosthesis (100) to match normal knee anatomy and eliminate the irritation to the muscles and soft tissues around the knee prosthesis (100).

It should be imperative that the construction and configuration of the device, system and any other elements or components described in the above detailed description should not be considered as a limitation with respect to the figures. Rather, variation to such structural configuration of the elements or components should be considered within the scope of the detailed description.

### EQUIVALENTS

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

While various aspects and embodiments have been disclosed herein, other aspects and embodiments will be apparent to those skilled in the art. The various aspects and embodiments disclosed herein are for purposes of illustration and are not intended to be limiting, with the true scope being indicated by the following claims.

Referral Numerals:

| Reference Number | Description |
|---|---|
| 100 | Knee prosthesis |
| 1 | Anterior flange |
| 2 | Posterior flange |
| 3 | Broad edge |
| 4 | Tapered flange |
| 6 | Patella orthopedic implant |
| 7 | Tibia orthopedic implant |
| 8 | Femoral orthopedic implant |
| 9 | Fat pad |
| 10 | Frictional contact area |
| 11 | Groove |
| 12 | Condylar member |
| 13 | First surface |
| 14 | Second surface |
| 15a, 15b | Posterior flange attachment |
| | members |
| 16 | Projection |
| 21 | Femur |
| 22 | Patella |
| 23 | Tibia |
| 24 | Flat plate |
| 25 | Protrusion |

1. A femoral orthopedic implant of a knee prosthesis, the femoral orthopedic implant comprising:
a condylar member;
a posterior flange extending away from one end of the condylar member and defined by a predetermined length; and
an anterior flange extending from another end, opposite and away from the one end of the condylar member, wherein the anterior flange is defined with a length ranging from about 0.75 to 1.4 times the predetermined length of the posterior flange.

2. The femoral orthopedic implant as claimed in claim 1, wherein the length of the anterior flange is equal to the predetermined length of the posterior flange.

3. The femoral orthopedic implant as claimed in claim 1, comprises a groove extending along a length of the condylar member and the anterior flange.

4. The femoral orthopedic implant as claimed in claim 1, wherein the posterior flange is defined with a pair of posterior flange attachment members extending parallel to each other from the condylar member.

5. The femoral orthopedic implant as claimed in claim 1, wherein the condylar member, the posterior flange and the anterior flange are integrally formed as a unitary structure.

6. The femoral orthopedic implant as claimed in claim 1, wherein the anterior flange and the posterior flange extend integrally from the condylar member defining a C-shaped or an U-shaped femoral orthopedic implant.

7. The femoral orthopedic implant as claimed in claim 1, wherein the condylar member, the posterior flange and the anterior flange comprises a first surface defined with a plurality of chamfers corresponding to a profile of the femur.

8. The femoral orthopedic implant as claimed in claim 1, comprises at least one projection configured to secure and lock the end of the femur and the femoral orthopedic implant.

9. A knee prosthesis, comprising:
a tibia orthopedic implant connectable to a top end of a tibia;
an insert connectable to the tibia orthopedic implant;
a patella orthopedic implant connectable to a patella; and
a femoral orthopedic implant, connectable to an end of a femur, the femoral orthopedic implant comprising:
a condylar member;
a posterior flange extending away from one end of the condylar member and defined by a predetermined length; and
an anterior flange extending from another end, opposite and away from the one end of the condylar member, wherein the anterior flange is defined with a length ranging from about 0.75 to 1.4 times the predetermined length of the posterior flange.

10. The knee prosthesis as claimed in claim 10, wherein the length of the anterior flange is equal to the predetermined length of the posterior flange.

11. The knee prosthesis as claimed in claim 10, wherein the condylar member, the posterior flange and the anterior flange are joined to form a unitary structure.

12. The knee prosthesis as claimed in claim 10, wherein the anterior flange and the posterior flange extend integrally from the condylar member defining a C-shaped or an U-shaped femoral orthopedic implant.

13. The knee prosthesis as claimed in claim 10, wherein the condylar member, the posterior flange and the anterior flange comprises a first surface defined with a plurality of chamfers corresponding to a profile of the femur.
